(19) United States
(12) Patent Application Publication
(10) Pub. No.: US 2011/0150997 A1
(43) Pub. Date: Jun. 23, 2011

(54) ELVUCITABINE PHARMACEUTICAL COMPOSITIONS

(75) Inventors: Gautam Shah, Westport, CT (US); Gary Bubb, Pittstown, NJ (US); Maryam Ahmadi, Newtown, CT (US)

(73) Assignee: ACHILLION PHARMACEUTICALS, INC., New Haven, CT (US)

(21) Appl. No.: 11/932,267

(22) Filed: Oct. 31, 2007

Related U.S. Application Data
(60) Provisional application No. 60/863,685, filed on Oct. 31, 2006.

(51) Int. Cl. A61K 31/7068; A61K 9/14; A61K 9/20; A61K 9/32; A61P 31/18

(52) U.S. Cl. 424/482; 514/49; 424/489; 424/494; 424/464; 424/490

(57) ABSTRACT

The invention provides novel pharmaceutical compositions of Elvucitabine. The invention also provides pharmaceutical compositions and oral dosage forms having unique physical-chemical properties.

### CROSS REFERENCE TO RELATED APPLICATION

This application claims priority from U.S. Provisional Application No. 60/863,685 filed Oct. 31, 2006, which is hereby incorporated by reference in its entirety.

### FIELD OF THE INVENTION

The invention provides novel pharmaceutical compositions comprising Elvucitabine. The invention also provides Elvucitabine pharmaceutical compositions and oral dosage forms having unique physical and chemical properties. The invention also provides methods of optimizing physical properties of compacted pharmaceutical compositions, including Elvucitabine tablet cores.

### BACKGROUND

Hepatitis B virus (HBV) infection is a major health problem throughout the world. HBV is a causative agent of both acute and chronic forms of hepatitis. It is estimated that more than 200 million people worldwide are chronic carriers of HBV.

HBV belongs to the family Hepadnaviridae, which includes a number of related viruses that primarily infect small rodents. All members of the hepadnavirus family have a number of characteristics in common such as morphological appearance, antigenic makeup and DNA size and structure. Pathological findings following infection with the members of this family are quite similar. Studies show that the replication and spread of the viruses of this family are dependent upon the reverse transcriptase of an RNA intermediate.

HBV is a double-stranded DNA virus. Its DNA polymerase catalyzes both DNA-dependent and RNA-dependent RNA synthesis. The life cycle of HBV involves the enzyme reverse transcriptase in its DNA replication.

Although acute HBV infections are generally self-limiting, in many instances the disease can progress to the chronic state. HBV infection also creates a risk to fulminant hepatitis. In addition, Hepatitis B viral infections are closely associated with hepatocellular carcinoma. There is presently no effective drug for the treatment of an HBV infection.

AIDS is a generally fatal disease caused by a human pathogenic retrovirus known as human T-lymphotropic virus type III (HTLV III), lymphadenopathy-associated virus (LAV) or human immunodeficiency virus (HIV). Reverse transcriptase plays an essential role in the elaboration and life cycle of HIV and consequently, the progress of the disease. Reverse transcriptase inhibitors are currently used with other classes of anti-viral agents to slow and in some cases halt the progress of HIV infection.

Reverse transcriptase inhibitors are preferred therapeutics for treating certain viral infections, particularly HBV and HIV infections. Typically, about 300 mg of a reverse transcriptase inhibitor must be administered daily for effective treatment of a viral infection, sometimes on a once per day dosing schedule, but more typically on a twice or three times per day dosing schedule. Because patients suffering from HBV or HIV often take a number of medications, a reverse transcriptase inhibitor efficacious at lower dosages is urgently needed. A reverse transcriptase inhibitor that can be administered once daily or less frequently is particularly desirable.

Elvucitabine is a nucleoside analog of the formula

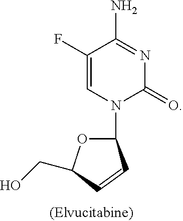

The anti-viral properties of Elvucitabine have been described previously in U.S. Pat. Nos. 5,621,120; 5,627,160; and 5,839,881, which are hereby incorporated by reference for their teachings regarding the use of Elvucitabine for treating viral infections, including HBV and HIV infections, and for teachings regarding the chemical synthesis of Elvucitabine.

Elvucitabine is acid labile and a difficult active pharmaceutical ingredient to formulate due, at least in part, to its limited compactability. Due to the moisture sensitivity and acid labile nature of Elvucitabine, a wet granulation process is likely not feasible. A direct compression process is preferable; however, not all commonly used direct compression excipients are compatible and suitable for use with Elvucitabine. The previous, mainly lactose based, direct compression Elvucitabine tablet formulation had a narrow range of compression force in which acceptable tablet cores with reasonable breaking force (hardness) and tablet strength can be produced that are suitable for coating. Excipients that are not acidic in nature, increase compactability, decrease tablet friability, are suitable for direct compression and provide stability for the Elvucitabine are desirable. It is also desirable to improve the blend and tablet dosage uniformity.

The present invention fulfills this need, and provides further related advantages.

### SUMMARY OF THE INVENTION

Provided herein are novel pharmaceutical compositions comprising Elvucitabine. In one embodiment, the invention provides a pharmaceutical composition comprising Elvucitabine and silicified microcrystalline cellulose (SMCC), a co-processed product composed of about 98 percent microcrystalline cellulose and about 2 percent colloidal silicon dioxide. A pharmaceutical composition comprising Elvucitabine, silicified microcrystalline cellulose and magnesium stearate, wherein about 0.05 to about 2% w/w of the composition is magnesium stearate is also provided herein. The invention also comprises a pharmaceutical composition comprising spray dried lactose and Elvucitabine and a pharmaceutical compositions comprising Elvucitabine, spray dried lactose, and SMCC.

The invention also provides Elvucitabine compositions having certain desirable physical and chemical properties. For example, a tablet comprising Elvucitabine and a pharmaceutically acceptable excipient, wherein the formulation has a tensile strength of about 2200 kPa to about 7700 kPa. A pharmaceutical composition in the form of a tablet containing about 2.5 mg Elvucitabine to about 20 mg Elvucitabine and having a breaking force, or breaking force, of about 55 Newtons to about 450 Newtons is also provided herein. The invention also provides an oral dosage form of Elvucitabine having a breaking force of about 90 Newtons to about 210 Newtons for 5 mg strength tablets and about 160 Newtons to about 340 Newtons for 10 mg strength tablets. The invention further provides a tablet core containing Elvucitabine, wherein the core has an inverse aspect ratio of 0.50 to 0.55. Furthermore, a pharmaceutical composition comprises Elvucitabine and at least one pharmaceutically acceptable excipient, wherein the formulation has compression of about 80 mPa to about 340 mPa.

A method of preparing a tablet core comprising Elvucitabine comprises reducing the particle size of an Elvucitabine sample to produce reduced particle size Elvucitabine, wherein the reduced particle size Elvucitabine comprises substantially all particles below 200 μm. In certain instances Elvucitabine synthesis produces a sample in which all the Elvucitabine particles are substantially below 200 μm. The Elvucitabine may then be used immediately after synthesis without further processing. Elvucitabine having a particle size of less than 200 μm and silicified microcrystalline cellulose are blended to form a blend comprising about 1% w/w to about 10% w/w of the Elvucitabine and about 30% w/w to about 95% w/w of the silicified microcrystalline cellulose, and compressing the blend to form a tablet core. Another method of preparing an Elvucitabine tablet core having a particle size of less than 200 μm comprises blending Elvucitabine and spray dried lactose to form a blend comprising about 1% w/w to about 10% w/w of the Elvucitabine and about 30% w/w to about 95% w/w of the spray dried lactose, and compressing the blend to form an Elvucitabine tablet core.

It has been discovered that Elvucitabine, a reverse transcriptase inhibitor, is efficacious for treating viral infections when administered at very low dosages. Furthermore, Elvucitabine is an effective anti-viral agent when as little as about 2.5 mg to about 20 mg is administered once a day. For some patients Elvucitabine may be effective when administered as infrequently as one time per week. Thus, methods of treating viral infections, including HIV and HBV infections, comprising administering about 2.5 mg to about 20 mg Elvucitabine per day, or about 5 mg to about 20 mg Elvucitabine per 48 hour interval, or about 15 mg to about 30 mg twice per week, or about 15 mg to about 60 mg Elvucitabine per week, or a loading dose of about 5 mg to about 40 mg followed by a daily dose or a less frequent dosing to a patient having a viral infection, such as an HIV or HBV infection, in which the Elvucitabine is in a formulation described herein, are within the scope of the invention.

Pharmaceutical compositions comprising about 2.5 mg to about 20 mg Elvucitabine, specifically about 5 mg to about 10 mg, are also within the scope of the invention. Particular Elvucitabine pharmaceutical compositions described herein are dose proportional, that is the same pharmaceutical composition can be used to prepare pharmaceutical dosage forms containing different amounts of Elvucitabine.

Packaged pharmaceutical compositions comprising an Elvucitabine pharmaceutical composition, in which the Elvucitabine is in a formulation described herein, and also comprising instructions for using the formulation for treating a viral infection according to the above-described dosage regimens to a patient suffering from a viral infection, are provided herein.

Elvucitabine compositions with enhanced mechanical strength are provided herein. Certain Elvucitabine compositions provided herein contain a blend of lactose and silicified microcrystalline cellulose (SMCC-90). Other Elvucitabine compositions provided herein contain a blend of spray-dried lactose and Elvucitabine.

### BRIEF DESCRIPTION OF THE DRAWINGS

FIG. 1. Conventional compaction profile data presentation. Data is presented as compression force vs. breaking force (hardness).

FIG. 2. Presentation of compaction profile data in which tablet breaking force is converted into a calculated tensile strength.

FIG. 3. Graph of the compacted formulation tensile strength as a function of the applied pressure.

FIG. 4. Relative size of the tablets made (after coating) as part of the tensile strength/applied pressure study

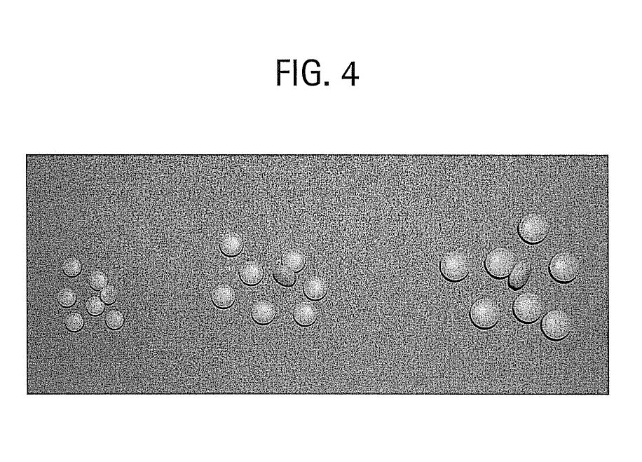

FIG. 5. Compaction Profiles for 77% lactose blend with 20% SMCC-90 (Prosolv 90)

FIG. 6. Compaction Profile Comparison of Spray Dried Lactose from Foremost Farms and Pharmatose

FIG. 7. Compaction Profiles for Different Ratios of Spray Dried and Anhydrous Lactose

FIG. 8. Compression event 1: 100% anhydrous lactose

FIG. 9. Compression event 2: 50% anhydrous lactose and 50% spray dried lactose

FIG. 10. Compression Event 3: 100% Spray Dried Lactose

FIG. 11. Ratios of SMCC-90 introduced into the Fast Flow 316 Blend

FIG. 12. Tensile strength of Lactose Fast Flow formulation (Optimized Lactose), Prosolv formulation (Optimized PROSOLV) and the Current Lactose Formulation.

### DETAILED DESCRIPTION OF THE INVENTION TERMINOLOGY

Prior to setting forth the invention in detail, it may be helpful to provide definitions of certain terms to be used herein. Unless defined otherwise, all technical and scientific terms used herein have the same meaning as is commonly understood by one of skill in the art to which this invention belongs.

The use of the terms “a”, “an” and “the” and similar referents in the context of describing the invention (especially in the context of the following claims) are to be construed to cover both the singular and the plural, unless otherwise indicated herein or clearly contradicted by context. The terms “comprising”, “having”, “including”, and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in a suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. “About” indicates an approximate amount, including the quantity it modifies. When “about” is used to modify a quantity of Elvucitabine, the free, unsalted form is the form of Elvucitabine referred to, unless another Elvucitabine form is expressly stated. No language in the specification should be construed as indicating any non-claimed element as essential to the practice of the invention.

The term “Elvucitabine” is meant to include solvates (including hydrates) of the free compound or salt, crystalline and non-crystalline forms, isotopically enriched or labeled forms, as well as various polymorphs of Elvucitabine, i.e. 4-amino-5-fluoro-14(2R,5S)-5-(hydroxymethyl)-2,5-dihydrofuran-2-yl)pyrimidin-2(1H)-one.

Elvucitabine contains asymmetric elements and can exist in different stereoisomeric forms. Elvucitabine can be, for example, a racemate or optically active form. While the 4-amino-5-fluoro-1-((2R,5S)-5-(hydroxymethyl)-2,5-dihydrofuran-2-yl)pyrimidin-2(1H)-one is preferred, methods of using racemic mixtures of Elvucitabine, and other optically pure stereoisomers of this compounds are within the scope of this invention. “Elvucitabine” particularly includes pharmaceutically acceptable salts of this compound.

Where an active agent exists in various tautomeric forms, the invention is not limited to any one of the specific tautomers, but rather includes all tautomeric forms.

Isotopes include those atoms having the same atomic number but different mass numbers. By way of general example, and without limitation, isotopes of hydrogen include tritium and deuterium and isotopes of carbon include ¹¹C, ¹³C, and ¹⁴C.

“Pharmaceutically acceptable salts” includes derivatives of Elvucitabine, wherein the parent compound is modified by making non-toxic acid or base addition salts thereof, and further refers to pharmaceutically acceptable solvates, including hydrates, of such compounds and such salts. Examples of pharmaceutically acceptable salts include, but are not limited to, mineral or organic acid addition salts of basic residues such as amines; alkali or organic addition salts of acidic residues such as carboxylic acids; and the like, and combinations comprising one or more of the foregoing salts. The pharmaceutically acceptable salts include non-toxic salts and the quaternary ammonium salts of the parent compound formed, for example, from non-toxic inorganic or organic acids. For example, non-toxic acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, and combinations comprising one or more of the foregoing salts.

Pharmaceutically acceptable organic salts include salts prepared from organic acids such as acetic, trifluoroacetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparginate, glutamate, and the like, and combinations comprising one or more of the foregoing salts.

The term “dosage form” denotes a formulation that contains an amount of Elvucitabine sufficient to achieve a therapeutic effect with a single or multiple administration. The term “oral dosage form” is meant to include a unit dosage form prescribed or intended for oral administration. An oral dosage form may or may not comprise a plurality of subunits such as, for example, microcapsules or microtablets, minicapsules or minitablets packaged for administration in a single dose. By “subunit” is meant to include a composition, mixture, particle, etc., that can provide an oral dosage form alone or when combined with other subunits. By “part of the same subunit” is meant to refer to a subunit comprising certain ingredients.

By “releasable form” is meant to include immediate-release, controlled-release, and sustained-release forms. Certain release forms can be characterized by their dissolution profile. Dissolution profile as used herein, means a plot of the cumulative amount of active ingredient released as a function of time. The dissolution profile can be measured utilizing the Drug Release Test <724>, which incorporates standard test USP 26 (Test <711>). A profile is characterized by the test conditions selected. Thus the dissolution profile can be generated at a preselected apparatus type, shaft speed, temperature, volume, and pH of the dissolution media.

Certain formulations described herein may be “coated”. The coating can be a suitable coating, such as, a functional or a non-functional coating, or multiple functional and/or non-functional coatings. By “functional coating” is meant to include a coating that modifies the release properties of the total formulation, for example, an enteric coating. By “non-functional coating” is meant to include a coating that is not a functional coating, for example, a cosmetic coating. A non-functional coating can have some impact on the release of the active agent due to the initial dissolution, hydration, perforation of the coating, etc., but would not be considered to be a significant deviation from the non-coated composition.

An “enteric coating” is a coating that prevents release of the active agent until the dosage form reaches the small intestine.

The term “effective amount” means an amount effective, when administered to a human or non-human patient, to provide a therapeutic benefit such as an amelioration of symptoms, e.g., an amount effective to decrease the symptoms of a viral infection, and preferably an amount sufficient to reduce the symptoms of an HBV or HIV infection. In certain circumstances, a patient suffering from a viral infection may not present symptoms of being infected. Thus, a therapeutically effective amount of a compound is also an amount sufficient to prevent a significant increase or significantly reduce the detectable level of virus or viral antibodies in the patient's blood, serum, or tissues. A significant increase or reduction in the detectable level of virus or viral antibodies is any detectable change that is statistically significant in a standard parametric test of statistical significance such as Student's T-test, where p<0.05.

The frequency of administration that will provide the effective results in an efficient manner without overdosing will vary with the characteristics of the particular formulation. Elvucitabine may be administered daily, every 48 hours, twice per week, or weekly, for example. In addition, a large loading dose may be employed followed by a lower dose administered daily or less frequently such as every 48 hours.

Administration of a particular dose includes administration of a single tablet, or multiple tablets, so long as the total amount of Elvucitabine is the amount desired for the total dose. For example, a 10 mg dose may be administered as 4×2.5 mg tablets, 2×5 mg tablets, or 1×10 mg tablet. Daily administration includes once, twice and thrice daily administration, for example. “BID administration” is twice daily administration of a compound, typically given during waking hours. “TID administration” is administration of a therapeutic compound three times daily, typically given during waking hours. Doses may also be given with or without food.

“Pharmacokinetic parameters” (PK) are parameters that describe the in vivo characteristics of Elvucitabine over time, including for example the in vivo dissolution characteristics and plasma concentration of active agent. By “Cₘₐₓ” is meant the measured concentration of Elvucitabine in the plasma at the highest observed concentration. By “C₂₄” is meant the concentration of Elvucitabine in the plasma at about 24 hours. The term “Tₘₐₓ” refers to the time at which the concentration of Elvucitabine in the plasma is the highest. “AUC” is the area under the curve of a graph of the concentration of Elvucitabine (typically plasma concentration) vs. time, measured from one time to another.

### Pharmacokinetics and Pharmacodynamics Modeling to Optimize the Therapeutic Index of Elvucitabine

Pharmacokinetics and pharmacodynamics modeling may be used to explore potential dosing regimens for Elvucitabine. A pharmacokinetics (PK) model was developed previously to describe both the plasma concentration-time data and excreted urinary amounts from previously completed studies with Elvucitabine (see U.S. patent application Ser. No. 11/547,179, which is hereby incorporated by reference for its teachings regarding pharmacokinetic models). With the PK model in hand and pharmacodynamics (PD) results of previously completed studies, the relationship of PK and PD from an efficacy and toxicity standpoint is obtained. From the results of the PK and PD studies, a therapeutic window of Elvucitabine is defined and simulated dosing regimens may be explored using ADAPT II® Pharmacokinetic and Pharmacodynamic Systems Analysis Software (Biomedical Simulations Resource, Los Angeles, Calif.). From the simulated dosing regimen results, dosing regimens may be identified that allow maintenance therapy with Elvucitabine at levels effective against both HIV and HBV while avoiding bone toxicity.

### Pharmaceutical Compositions

Provided herein are novel Elvucitabine compositions, including those suitable for low dosage and/or low frequency administration, comprising Elvucitabine or a salt thereof and at least one excipient such as a filler and/or binder. In one embodiment, the filler comprises silicified microcrystalline cellulose. The filler may additionally comprise of SMCC, a combination of SMCC and spray dried lactose, or spray dried lactose alone. The pharmaceutical composition may be in a form suitable for administration to a human subject, for example, but is preferably an oral dosage form such as a tablet or capsule. The formulations provided herein may be formulated by a variety of methods apparent to those of skill in the art of pharmaceutical composition.

In one embodiment, the Elvucitabine has substantially all particles less than 200 microns, or 90% of the particles between about 50 and about 150 microns, or 98% of the particles between about 25 and about 180 microns. The particle size of Elvucitabine may optionally be reduced using a suitable method such as milling or micronizing with, for example, a jet mill, so long as the desired particle size is produced. The Elvucitabine particle size reduction may be performed prior to blending the Elvucitabine with the excipients, or may be performed on a blend.

In certain embodiments, the Elvucitabine compositions comprise silicified microcrystalline cellulose (SMCC). An exemplary silicified microcrystalline cellulose is PROSOLV SMMC 90, JRS Pharma LP, Patterson, N.Y. (herein after Prosolv 90), a product composed of about 98 percent microcrystalline cellulose and about 2 percent colloidal silicon dioxide. Silicification of the microcrystalline cellulose is achieved through a process that results in an intimate association of the colloidal silicon dioxide and microcrystalline cellulose. The median particle size of PROSOLV 90 silicified microcrystalline cellulose is about 90 μm. Another suitable silicified microcrystalline cellulose is a silicified microcrystalline cellulose having a median particle size of about 50 μm, e.g. PROSOLV 50.

The pharmaceutical composition formulation may additionally comprise one or both of lactose and silicified microcrystalline cellulose having a median particle size of approximately 50 p.m. For example, included within this invention are SMCC containing Elvucitabine compositions, as described about in which up to about 25% w/w of the SMCC having a median particle size of about 90 μm is replaced by SMCC having a median particle size of about 50 μm. In other embodiments about 30-60% w/w of the SMCC may be replaced by lactose, such as lactose monohydrate.

The Elvucitabine composition may be in the form of a core, such as a pressed core or a pellet, which is optionally coated. As used herein, the term core refers to an Elvucitabine composition absent any coatings. When the Elvucitabine composition comprises silicified microcrystalline cellulose, the core comprises about 1% w/w to about 10% w/w of the Elvucitabine and about 30% w/w to about 95% w/w of the silicified microcrystalline cellulose, based on the total weight of the core composition. In another embodiment, the core comprises about 1% w/w to about 10% w/w of the Elvucitabine and about 86% w/w to about 95% w/w of the silicified microcrystalline cellulose, based on the total weight of the core composition. In some embodiments described herein the composition comprises about 90 to about 95% w/w silicified microcrystalline cellulose.

The Elvucitabine compositions may comprise pharmaceutically compatible excipients. Excipients may be added to facilitate manufacture, enhance stability, control release, enhance product characteristics, enhance bioavailability, enhance patient acceptability, etc. Suitable excipients include inert diluents, such as calcium carbonate, sodium carbonate, mannitol, lactose, cellulose, and combinations comprising one or more of the foregoing diluents; binders such as starch, methylcellulose, gelatin, sucrose, and combinations comprising one or more of the foregoing binders; disintegrants such as starch, alginic acid, sodium starch glycolate, croscarmelose, and combinations comprising one or more of the foregoing disintegrants; and lubricants such as magnesium stearate, stearic acid, talc, sodium stearyl fumarate (e.g. PRUV) and combinations comprising one or more of the foregoing lubricants. Agents such as potassium phosphate, magnesium oxide, potassium citrate, and sodium phosphate may also be present. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules (including time release and sustained release formulations) typically comprise one or more solid diluents disclosed above. The selection of carrier components often depends on secondary considerations like taste, cost, and shelf stability.

In one embodiment, the additional excipients comprise about 0.25% w/w to about 3% w/w of a disintegrant (e.g., sodium starch glycolate), about 1% w/w to about 5% w/w of a buffering agent (e.g., potassium phosphate, calcium silicate), about 0.05% w/w to about 2% w/w, specifically about 0.05% w/w to about 0.75% w/w of a lubricant (e.g., magnesium stearate), and combinations comprising one or more of the foregoing additional excipients.

### Preparation of Cores

Cores comprising Elvucitabine can be prepared by various mixing, comminution and fabrication techniques readily apparent to those skilled in the area of drug formulations. Examples of such techniques are as follows

(1) Direct compression, using appropriate punches and dies; the punches and dies are fitted to a suitable rotary tablet press;

(2) Injection or compression molding using suitable molds fitted to a compression unit; and

(3) Granulation followed by compression.

When cores are made by direct compression, the addition of lubricants may be helpful to promote powder flow and to reduce capping of the particle (breaking off of a portion of the particle) when the pressure is relieved. Useful lubricants include, for example, magnesium stearate in a concentration of about 0.05% to about 2% by weight, preferably less than about 1% by weight, in the powder mix. Additional excipients may be added to enhance powder flowability and reduce adherence.

Prior to compression, the Elvucitabine, filler, lubricant and additional excipients are blended for a time sufficient to produce the desired pressed product. Blending times may be about 15 to about 120 minutes. The composition and process here describes use of less than 2.0% lubricant, and in most embodiments less than 0.75% lubricant. The lubricant is added at the beginning of the blending process or early in the blending process and blended for at least 15 minutes with the Elvucitabine. This method allows for uniform distribution of lubricant in the blend, reaching equilibrium and is not expected to adversely impact the compaction profile of the tablet blend upon further mixing, either in a blender or on a tablet press.

### Preparation of Elvucitabine Containing Subunits

Elvucitabine and any optional additives may be prepared in many different ways, for example as subunits. Pellets comprising an active agent can be prepared, for example, by a melt pelletization technique. In this technique, the active agent in finely divided form is combined with a binder and other optional inert ingredients, and thereafter the mixture is pelletized, e.g., by mechanically working the mixture in a high shear mixer to form the pellets (e.g., pellets, granules, spheres, beads, etc., collectively referred to herein as “pellets”). Thereafter, the pellets can be sieved in order to obtain pellets of the requisite size. The binder material may also be in particulate form and has a melting point above about 40° C.

Subunits, e.g., in the form of multiparticulates, can be compressed into an oral tablet using conventional tableting equipment using standard techniques. Alternatively, subunits may be in the form of mini-tablets or micro-tablets enclosed inside a capsule, e.g., a gelatin capsule. For this, a gelatin capsule as is employed in pharmaceutical compositions can be used, such as the hard gelatin capsule known as CAPSUGEL, available from Pfizer.

### Preparation of Lactose Containing Cores

An improvement in tablet strength of existing Elvucitabine lactose formulations was needed. The core tablets had marginally acceptable friability only within a narrow range of compression force, with propensity to cap under higher compression forces. SMCC-90 (20%) was added to a lactose-based Elvucitabine core formulation in effort to enhance the mechanical strength of the compacted formulation. This formulation did not show improved strength. The lactose portion of the formulation comprised of equal parts of spray dried and anhydrous lactose.

A series of tests followed that demonstrated the relative strengths of different grades of lactose and lactose/SMCC blends. Different ratios of the spray dried and anhydrous lactose were compacted and evaluated on an Riva Piccola tablet press. Likewise, compaction profiles of the spray-dried lactose from different manufacturers were evaluated. Finally, SMCC-90 was gradually re-introduced into the preferred lactose blend with good results.

The inventors have discovered that spray dried lactose from two different manufactures showed significantly different strength and capping potentials. Spray dried lactose alone provided more strength than any lactose ratio using anhydrous lactose, and SMCC addition improved the strength of spray dried lactose, but had little effect on a 50/50 lactose blend.

### Physical and Chemical Properties of Tablets

Pharmaceutical compositions for use in tablets may have certain desirable physical properties. For example the invention provides a pharmaceutical composition of Elvucitabine having a tensile strength greater than 2100 kPa, or in other embodiments having a tensile strength of about 2200 kPa to about 7700 kPa. Also provided is pharmaceutical composition of Elvucitabine having a compression range of about 80 mPa to about 350 mPa.

A pharmaceutical composition in the form of a tablet containing about 2.5 mg Elvucitabine to about 20 mg Elvucitabine and having a breaking force, or breaking force, of about 55 Newtons to about 450 Newtons is also provided herein. The invention also provides an oral dosage form of Elvucitabine having a breaking force of about 90 Newtons to about 210 Newtons for 5 mg strength tablets and about 160 Newtons to about 340 Newtons for 10 mg strength tablets. The invention further provides a tablet core containing Elvucitabine, wherein the core has an inverse aspect ratio of 0.50 to 0.55. Furthermore, a pharmaceutical composition comprises Elvucitabine and at least one pharmaceutically acceptable excipient, wherein the formulation has compression of about 80 mPa to about 340 mPa.

The invention includes an Elvucitabine oral dosage form, in which the Elvucitabine is in a formulation as described herein, for example a tablet, wherein the core has an inverse aspect ratio of 0.45 to 0.6 (0.50 to 0.55 is preferred for some embodiments). The invention includes Elvucitabine, wherein the coated tablet has an inverse aspect ratio of 0.45 to 0.6 (0.51 to 0.56 is preferred for some embodiments).

The invention includes methods of optimizing physical properties of compacted pharmaceutical compositions, including Elvucitabine tablet cores. Measurement of compaction profiles is a very effective tool in formulation development and understanding the physical properties of a compact when compressed on a rotary tablet press. Conventionally the data is presented in a graphical format showing the relationship between the applied compression force and the resulting tablet breaking force (hardness). This relationship changes for each tablet geometry. The inventors have discovered that a better and more meaningful approach in understanding the compaction properties of a granulation for different tablet size and strengths is the conversion of the applied compression force into a compression pressure and the tablet breaking force into a tensile strength. In other words, the inventors have determined that normalizing data obtained from a rotary tablet press is desirable.

The equation conventionally used for calculating the tensile strength of a flat faced cylindrical core is:

Tensile strength=(2)(breaking force)/(π)(breaking area) (Equation 1)

Where breaking area=diameter×thickness

This equation was originally derived by the Russian mathematician S. P. Timoshenko subsequently evolved into an American Society for Testing and Materials Standard, ASTM C-496 and is primarily used for testing concrete cores.

The diametrical compression test (hardness tester) is generally used to measure the force required to break a compact. Provided that the compact is round with flat faces and fails in a direction perpendicular to the axis of the load the above method can be used to compute the tensile strength of the compact. Localized compression (crushing) failure or shear failure will not appreciably affect the results unless it represents more than 10% of the diameter.

Another limitation for the application of the above equation is that the compression strength must be at least three times higher than the tensile strength. This is generally not an issue in the pharmaceutical industry as most powders made into a compact exhibit a tensile strength less than 20% of the compressive strength.

In 1989 J. M. Newton developed the following equation with the help of photoelasticity to determine the tensile strength of convex shaped tablets. This equation was used in herein for understanding the compaction properties a compacted pharmaceutical granulation for different tablet size and strengths.

$${σ}_{t} = \frac{10 F}{π {D}^{2}} {( 2.84 \frac{t}{D} - 0.126 \frac{t}{{C}_{L}} + 3.15 \frac{{C}_{L}}{D} + 0.001 )}^{- 1} \qquad ( Equation 2 )$$

Wherein: σₜ=tensile strength
F=Breaking force (hardness)

### D=Tablet Diameter

t=Overall tablet thickness
C_{L}=Tablet belly band length.
MCC and Lactose based formulations have been very successful normalized via this method.

This method provides several advantages for determining compaction properties of compacted pharmaceutical compositions over conventional methods. The method permits the use of smaller tooling requiring much less material, which is desirable when expensive active agents are tableted. The method increases provides better understanding of tablet physical properties. Tablet breaking force and compression force for each tablet geometry is different. A suitable breaking force for one tablet might not be acceptable for another tablet. Compressing to a given tensile strength might work for all tablet sizes may provide desirable physical properties for all tablet sizes and geometries. The tensile strength takes into account the tablet geometry. Applied force is converted into applied pressure based on the cross sectional area. Compressing to a given pressure eliminates the guesswork of what force is needed for a particular size tablet. The method reduces the number of tablets required, thereby reducing equipment utilization time, not only with the tablet press, but all associated support equipment and personnel. This method also reduces the number of tablets required for a dose proportional formulation and is more cost effective to produce.

The inventors have also investigated whether capping varies as a function of tablet size. To investigate, the ratios of the areas involved for tensile strength and cross sectional were determined for each tablet size. The tensile strength area is dependent on the diameter and tablet thickness, while the cross sectional area for capping is dependent on the diameter squared. The ratio will be the tensile strength area/capping strength area. Tablets with a smaller ratio, therefore have a greater propensity towards capping.

Tensile Strength is proportional to 1/φt where φ is the diameter and t is the thickness.

Cross Sectional Area is proportional to φ². The ratio therefore becomes φ/t which is the same as the aspect ratio. Only the largest tablets were found to have a greater propensity to capping.

### Coatings

The formulations described herein may be coated with a functional or non-functional coating.

In one embodiment, a functional coating comprises an enteric coating polymer. The enteric polymer should be non-toxic and is predominantly soluble in the intestinal fluid, but substantially insoluble in the gastric juices. Examples include polyvinyl acetate phthalate (PVAP), hydroxypropylmethyl-cellulose acetate succinate (HPMCAS), cellulose acetate phthalate (CAP), methacrylic acid copolymers, hydroxy propyl methylcellulose succinate, cellulose acetate succinate, cellulose acetate hexahydrophthalate, hydroxypropyl methylcellulose hexahydrophthalate, hydroxypropyl methylcellulose phthalate (HPMCP), cellulose propionate phthalate, cellulose acetate maleate, cellulose acetate trimellitate, cellulose acetate butyrate, cellulose acetate propionate, methacrylic acid/methacrylate polymer (acid number 300 to 330 and also known as EUDRAGIT L), which is an anionic copolymer based on methacrylate and available as a powder (also known as methacrylic acid copolymer, type A NF, methacrylic acid-methyl methacrylate copolymer, ethyl methacrylate-methylmethacrylate-chlorotrimethylammonium ethyl methacrylate copolymer, and the like, and combinations comprising one or more of the foregoing enteric polymers. Other examples include natural resins, such as shellac, SANDARAC, copal collophorium, and combinations comprising one or more of the foregoing polymers. Yet other examples of enteric polymers include synthetic resin bearing carboxyl groups. A suitable methacrylic acid copolymer is commercially available as Acryl EZE®. The methacrylic acid:acrylic acid ethyl ester 1:1 copolymer solid substance of the acrylic dispersion sold under the trade designation “EUDRAGIT L-100-55” may be suitable.

The enteric coating may comprise about 8 w/w to about 25% w/w, or about 8% w/w to about 16% w/w of the total weight of the dosage form.

The dosage forms may comprise additional functional and nonfunctional coatings such as seal coats and overcoats. In one embodiment the coating is a color coating. OPADRY orange is an example of a suitable color coating. In some embodiments an enteric coating is subsequently color coated. The coating material may include a polymer, preferably a film-forming polymer, for example, methyl cellulose, ethyl cellulose, hydroxypropyl cellulose, hydroxypropyl methyl cellulose, hydroxybutyl methyl cellulose, cellulose acetate, cellulose propionate (lower, medium or higher molecular weight), cellulose acetate propionate, cellulose acetate butyrate, cellulose acetate phthalate, carboxymethyl cellulose, cellulose triacetate, cellulose sulphate sodium salt, poly(methyl methacrylate), poly (ethyl methacrylate), poly (butyl methacrylate), poly (isobutyl methacrylate), poly (hexyl methacrylate), poly (phenyl methacrylate), poly (methyl acrylate), poly (isopropyl acrylate), poly (isobutyl acrylate), poly (octadecyl acrylate), poly (ethylene), poly (ethylene) low density, poly (ethylene) high density, (poly propylene), poly (ethylene glycol poly (ethylene oxide), poly (ethylene terephthalate), poly(vinyl alcohol), poly(vinyl isobutyl ether), poly(vinyl acetate), poly (vinyl chloride), polyvinyl pyrrolidone, and combinations comprising one or more of the foregoing polymers. A suitable hydroxypropyl methylcellulose is commercially available as OPADRY from Colorcon.

A seal coat and/or an overcoat may comprise about 1% w/w to about 5% w/w of the total weight of the dosage form.

The inclusion of an effective amount of a plasticizer in the coating composition may improve the physical properties of the film. Generally, the amount of plasticizer included in a coating solution is based on the concentration of the polymer, e.g., most often from about 1 to about 50 percent by weight of the polymer. Concentrations of the plasticizer, however, can be determined by routine experimentation.

Examples of plasticizers for ethyl cellulose and other celluloses include plasticizers such as dibutyl sebacate, diethyl phthalate, triethyl citrate, tributyl citrate, triacetin, and combinations comprising one or more of the foregoing plasticizers, although it is possible that other water-insoluble plasticizers (such as acetylated monoglycerides, phthalate esters, castor oil, etc.) can be used.

Examples of plasticizers for acrylic polymers include citric acid esters such as triethyl citrate 21, tributyl citrate, dibutyl phthalate, 1,2-propylene glycol, polyethylene glycols, propylene glycol, diethyl phthalate, castor oil, triacetin, and combinations comprising one or more of the foregoing plasticizers, although it is possible that other plasticizers (such as acetylated monoglycerides, phthalate esters, castor oil, etc.) can be used.

Suitable methods are used to apply the coating to tablet cores, for example. Processes such as simple or complex coacervation, interfacial polymerization, liquid drying, thermal and ionic gelation, spray drying, spray chilling, fluidized bed coating, pan coating, electrostatic deposition, may be employed. A substantially continuous nature of the coating may be achieved, for example, by spray drying from a suspension or dispersion of active agent in a solution of the coating composition including a polymer in a solvent in a drying gas having a low dew point.

When a solvent is used to apply the coating, the solvent may be water or an organic solvent. The solvent may constitute a good solvent for the coating material, but is substantially a non-solvent or poor solvent for an active agent. The solvent may be selected from water, alcohols such as methanol, ethanol, halogenated hydrocarbons such as dichloromethane (methylene chloride), hydrocarbons such as cyclohexane, acetone, and combinations comprising one or more of the foregoing solvents.

The concentration of polymer in the solvent will normally be less than about 75% w/w, and typically about 10% w/w to about 30% w/w. After coating, the coated dosage forms may be allowed to cure for about 1 to about 2 hours at a temperature of about 50° C. to about 60° C., more preferably of about 55° C.

The coatings may be about 0.005 μm to about 25 μm thick, preferably about 0.05 to about 5 p.m.

### Capsules

the Elvucitabine, Optionally in the Form of Pellets, May be Disposed in a Capsule. The capsule may comprise a hard capsule and/or soft capsule. A hard capsule may be composed of two parts, a cap and a body, which are fitted together after the larger body is filled with the active agent. The hard capsule may be fitted together by slipping or telescoping the cap section over the body section, thus completely surrounding and encapsulating the active agent. A soft capsule may be a one-piece soft capsule of sealed construction encapsulating the active agent. The soft capsule may be made by various processes, such as the plate process, the rotary die process, the reciprocating die process, and the continuous process. A gelatin capsule as is employed in pharmaceutical compositions can be used, such as the hard gelatin capsule known as CAPSUGEL, available from Pfizer.

### Dosage Forms Characterized by AUC

The Elvucitabine compositions described herein exhibit characteristic plasma concentrations over time. When integrated the graph of plasma concentration over time provides a characteristic “area under the curve” or AUC.

Certain Elvucitabine dosage forms described herein may exhibit an AUC at steady state for a 24 hour period of about 300 microgram hour/liter (μg h/L).

### Packaged Formulations

Packaged pharmaceutical compositions comprising an Elvucitabine pharmaceutical composition and instructions for using the formulation for treating a viral infection, such as an HBV or HIV infection, to a patient suffering from a viral infection, are further provided herein. The instructions may comprise administration instructions as described in “Methods of Treatment” below. Typically the instructions will be instructions for using the formulation to treat an HBV or HIV infection. Packaged formulations in which the Elvucitabine is present as an oral dosage form are included.

The invention includes providing prescribing information, for example, to a patient or health care provider, or as a label in a packaged pharmaceutical composition. Prescribing information may include for example efficacy, dosage and administration, contraindication and adverse reaction information pertaining to the pharmaceutical composition.

### Methods of Treatment

Provided herein is a method of treating a viral infection, such as an HBV or an HIV infection, comprising administering Elvucitabine in a formulation as described herein, to a patient suffering from a viral infection. In one embodiment, administration is once per day, and the dosage is 2.5 mg to 20 mg per day, specifically 2.5 mg to 15 mg per day, and more specifically 2.5 mg to 10 mg per day.

In another embodiment, the Elvucitabine composition is administered once every 48 hours and the dosage is 5 mg to 20 mg every 48 hours, specifically 5 mg to 10 mg every 48 hours.

In yet another embodiment, the Elvucitabine composition is administered twice per week, and the dosage is 30 mg to 50 mg per dose

In another embodiment, the Elvucitabine is administered once per week, and the dosage is 15 mg to 60 mg per week.

In certain embodiments, administration may comprise administration of a loading dose. A loading dose, as used herein, is a quantity higher than the average or maintenance dose, used at the initiation of therapy to rapidly establish a desired level of the Elvucitabine. A loading dose may comprise about 5 to about 40 mg of Elvucitabine. The loading dose is followed by a maintenance dose of Elvucitabine which is lower than the loading dose.

Also provided herein are methods of increasing patient compliance with anti-viral therapy, such as treatment of an HIV or HBV infection, by providing Elvucitabine, in which the Elvucitabine is in a formulation as described herein. A dosage form to increase patient compliance may be formulated for low dose daily administration. Methods of increasing patient compliance with anti-HIV therapy by providing Elvucitabine formulated for administration of about 2.5 to about 20 mg of the Elvucitabine daily are included in the invention. In certain embodiments, the anti-viral therapy is treatment of an HBV or HIV infection.

The following examples further illustrate the invention but should not be construed as in any way limiting its scope.

### EXAMPLES

### Abbreviations

The following abbreviations are used in the examples, which follow, and elsewhere. This list is not meant to be an all-inclusive list of abbreviations used in the application as additional standard abbreviations, which are readily understood by those skilled in the art of pharmaceutical compositions, may also be used. It should be noted that the processing parameters provided are those used for relatively small formulation batches of 3-4 kg. Those of skill in the art of pharmaceutical composition will recognize that processing parameters will need to be adjusted for other batch sizes; such adjustments are routine within the art.

### cfm Cubic Feet per Minute

h hour

### kPa Kilo Pascal

mPa mega Pascal
NMT Not more than

### Example 1

### Particle Size Reduction of Elvucitabine

Prior to tablet core manufacture, Elvucitabine particle size may be reduced, using either a milling or micronization process. Here, micronization refers to the process of creating smaller, more uniform particles, via a micronization method rather than making micro-particles of 5-10 microns. Micronization may be achieved by a standard micronization procedure for preparation of active pharmaceutical ingredients. For example, micronization may be via a jet-mill process. Parameters are as follows: Injection pressure, 2-8 kg/cm2; micronization pressure, 5-15 kg/cm2; and cyclone pressure, 1-5 kg/cm2. Particle size may be determined by laser light scattering or by sieve analysis, for example. (See U.S. Pat. No. 6,852,737 at Col. 29, lines 31-45, which is hereby incorporated by reference for its teachings regarding micronization of an active pharmaceutical ingredient.)

The particle size reduction of Elvucitabine using a micronization process may be achieved using an eight inch spiral jet mill with nitrogen gas.

Elvucitabine particles created by these micronization processes are less than 200 microns in size.

Alternatively, Elvucitabine may first be ground using a knife-grinder and then micronized in an ALJET Micronizer (FLUID ENERGY ALJET, Plumsteadsville, Pa., USA) using a pressurized dry nitrogen stream. (See U.S. Pat. No. 6,555,156, at Col. 6, lines 13-18, which is hereby incorporated by reference for its teachings regarding micronization of an active pharmaceutical ingredient).

The mean particle size of the Elvucitabine after micronization is substantially all particles below 210 microns, or 90% of particles between 50 and 150 microns, or 98% of the particles between 25 and 180 microns.

### Example 2

### Optimization of Tablet Hardness

Three different strengths of low dose tablets using a common blend were desired. Tablets and tooling were designed to result in total tablet weights of 75, 150, and 300 mg representing the three dosage strengths. A compound radius design was used on the tooling as the final product was to be coated. Compaction profiles and strain rate studies were performed and the results graphically presented.

The following materials and machines shown in Tables 1 and 2 were used in the experiments.

TABLE 1
TABLET CORE

| Blend Component | % w/w |
|---|---|
| Silicified Microcrystalline Cellulose, NF | 92.57 |
| (Prosolv 90) | |
| Explotab | 2.00 |
| Dipotassium Phosphate Powder, USP, PE | 2.00 |
| Magnesium Stearate, NF, EP (VG) | 0.10 |
| Elvucitabine, micronized | 3.33 |
| Totals | 100.0 |

TABLE 2
INSTRUMENTATION

| | |
|---|---|
| Blender | Turbula T2F (Glen Mills) |
| Tablet Press | Riva Piccola 10 station instrumented tablet press |
| | distributed by SMI |
| Tooling | Three sets of TSM domed B tooling using a |
| | double compound radius suitable for making the |
| | three sizes; 75 mg; 150 mg and 300 mg tablets |
| Instrumentation | SMI “The Director” |
| System | |
| Hardness Tester | VK-200 (Van Kel) |

Riva Piccola instrumented tablet press was used in the studies to determine the ejection, take-off and compaction forces. The SMI Director program was used to acquire and generate the graphs and reports shown in this Example. The relative size of the tablets made (after coating) as part of this study.

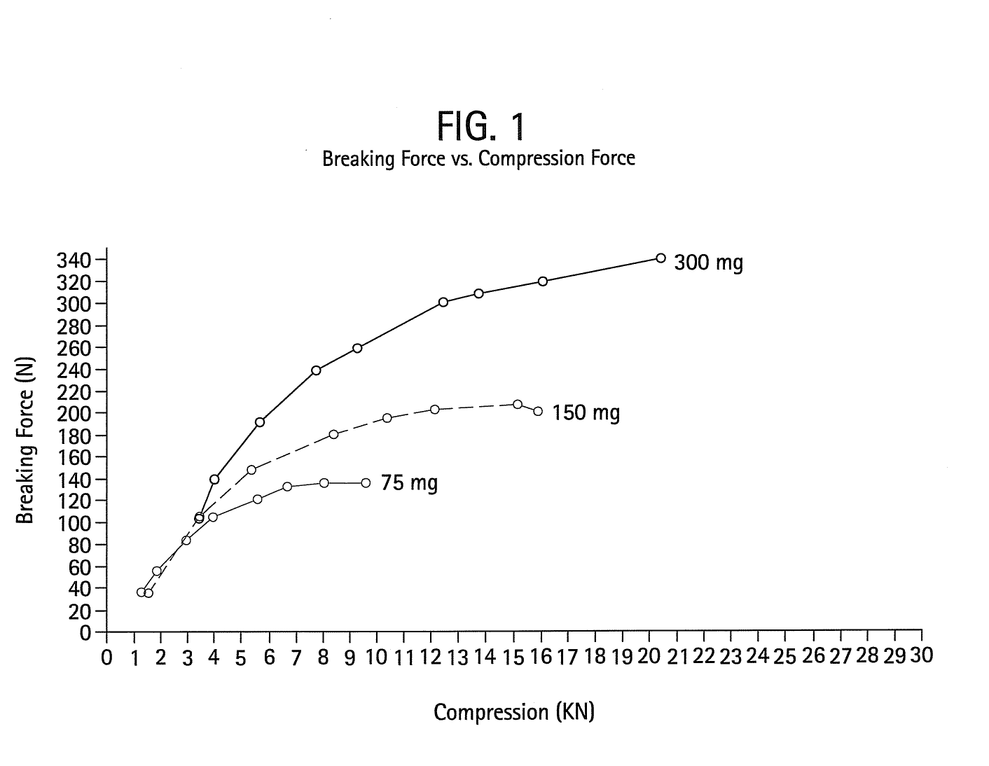

All the results are shown graphically for ease in interpretation and clarity. The graph of FIG. 1 shows how the data is normally presented, that is compression force vs. breaking force (hardness). The graphical representation demonstrates that this relationship does not allow meaningful evaluation of the mechanical properties of the formulation and varying tablet size and shapes. The graph provided in FIG. 1 represents three different tablet sizes made from exactly the same formulation. As one would expect it takes more force to make a larger tablet and it takes more force to break a larger tablet even though the core formulation is identical.

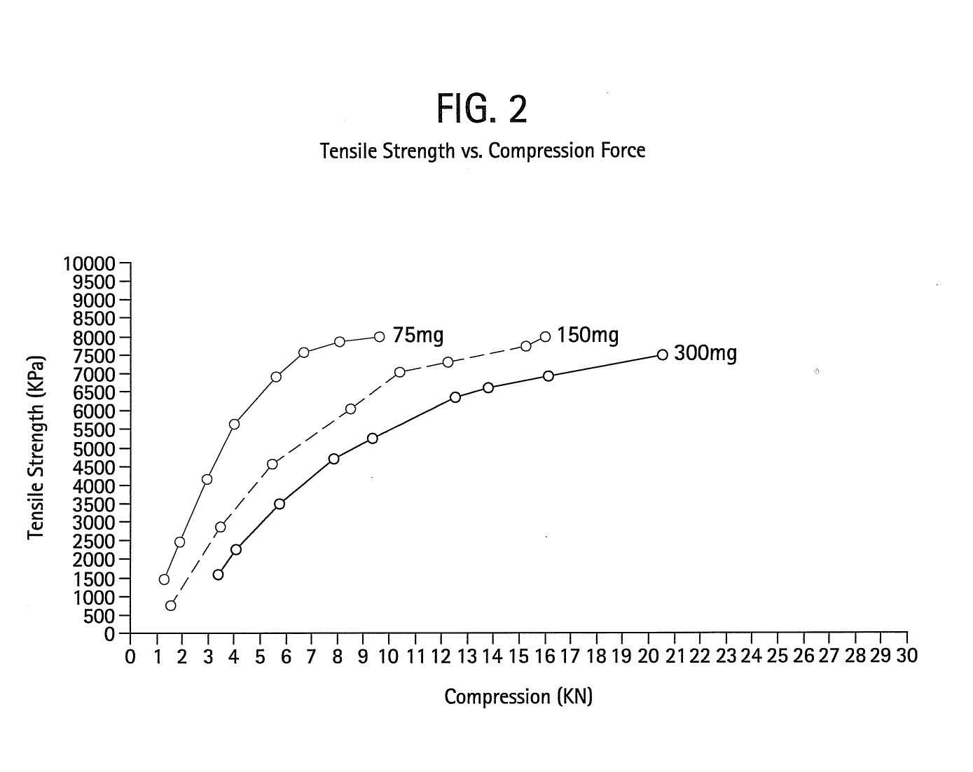

FIG. 2 shows a conversion of the tablet breaking force into a calculated tensile strength. When the breaking force is converted into tensile strength it becomes clear that for the same force a smaller tablet is actually stronger than the larger one, even though it takes more force to break the larger one. From the standpoint of mechanical strength, the smaller tablet, made at the same force is stronger.

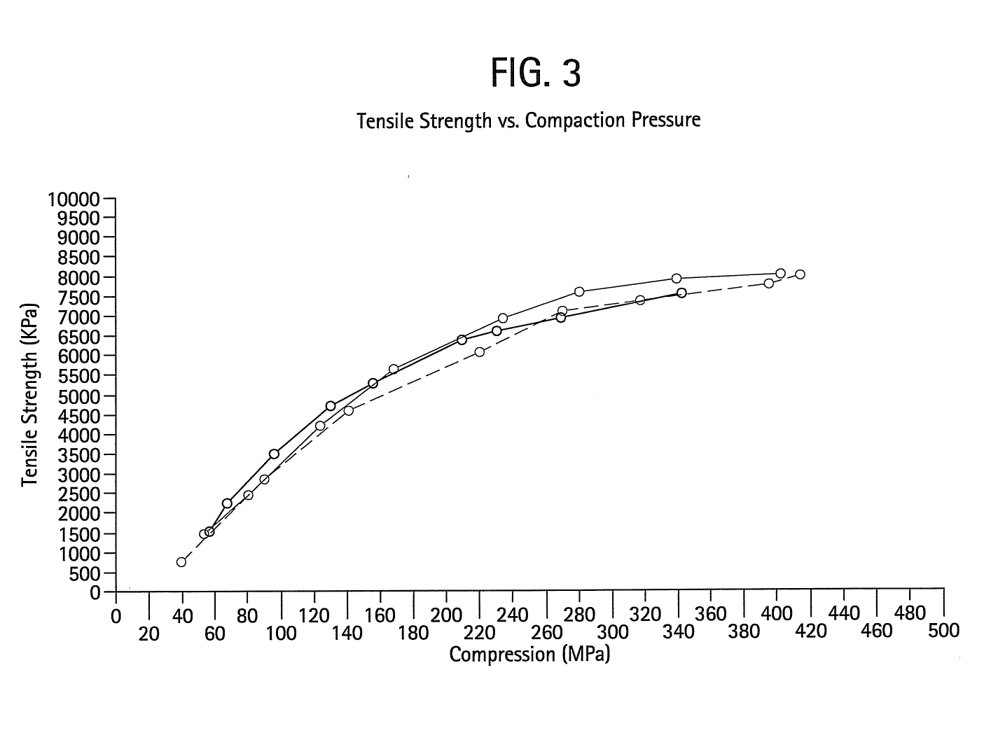

FIG. 3 provides a plot of the tensile strength as a function of the applied pressure. The data in FIG. 3 demonstrate that by using tensile strength and applied pressure the tablet geometry is effectively taken out of the equation and the results are normalized. The tensile strength takes into account the tablet geometry and the applied force is converted into applied pressure based on the cross sectional area. This allows one to truly study the mechanical properties of the formulation and not be mislead by varying tablet sizes and shapes.

When completely normalized the 75 mg; 150 mg, and 300 mg tablet data superimpose. The data in this fashion allows for a more meaningful and predictable evaluations and eliminates the need for multiple tests on each different size/strengths of tablet. This in turn can be expected to shorten the development time and minimize the burden on API in the early phases of development when the API supply is limited. In this instance all of the testing for all three tablet sizes was performed. It would have been feasible to select only one geometry for the formulation development, then as a final check before clinical trials make tablets for each geometry at the calculated forces that represents the desired pressures.

### Example 3

### Determination of Capping as a Function of Tablet Size

As discussed above a decreased ratio of diameter/thickness increased tendency towards capping. The data shown in TABLE 3 indicates that only the largest tablet size showed any tendency towards capping and then only at the highest pressures.

TABLE 3

| | | | Ratio |
|---|---|---|---|
| Size (mg) | Diameter (mm) | Thickness (mm) | (diameter/thickness) |
| 75 | 5.5 | 2.75 | 2.00 |
| 150 | 7.0 | 3.55 | 1.97 |
| 300 | 8.73 | 4.65 | 1.88 |

### Example 4

### Elvucitabine Common Blend for Enteric Coated Tablets

Blend components are added according to Table 4.

TABLE 4

| Blend Component | Weight | % w/w |
|---|---|---|
| Silicified Microcrystalline Cellulose, NF | 10.645 kg | 92.57 |
| (Prosolv 90) | | |
| Sodium Starch Glycolate, NF, EP | 0.230 kg | 2.00 |
| Dipotassium Phosphate Powder, USP, PE | 0.230 kg | 2.00 |
| Magnesium Stearate, NF, EP (VG) | 0.012 kg | 0.10 |
| Elvucitabine, micronized | 0.383 kg | 3.33 |
| Totals | 11.5 kg | 100.0 |

Prosolv 90, micronized Elvucitabine, and Dipotassium Phosphate Powder, are added sequentially into a Bohle Bin Blender (BL07C, Warminster, Pa., USA) and blended for 10±0.1 minutes at 11±1 rpm. Additional Prosolv 90, Sodium Starch Glycolate, NF, EP, and Magnesium Stearate, NF, EP (VG) are added and blended for 10±0.1 minutes at 11±1 rpm. The material is then milled in a Bohle In-Line High Speed Mill (ML19) or equivalent and then passed through a 0.5 mm screen (35 Mesh) operated at a speed of 1400 rpm±50 rpm. Additional Prosolv 90 is passed through a Bohle In-Line High Speed Mill (ML19) and then passed through a 0.5 mm Screen (35 Mesh) operated at a speed of 1400±50 rpm.

The 40 liter Bohle Bin Blender (BL07C) is charged with the milled materials. The blender bin cover is secured and the components are blended for about 60 minutes at 11±1 rpm. Particle size analysis and bulk density analysis may be performed on the blend.

### Example 5

### Preparation of Enteric Coated Elvucitabine Tablets (5 mg Strength)

### Example 5a

### Manufacture of Tablet Cores

A 11.500 kg Elvucitabine common blend, prepared as described in Example 2, is loaded into a tablet compressing machine, such as a Fette 1200 B Tool Tablet Press (TP06) or equivalent, and tablets are formed using 0.2756″ round plain upper and lower punches. Tablets are obtained having an average tablet weight of 150.0 mg with average acceptable upper and lower tablet weight limits of 5.0% (142.5 mg to 157.5 mg) and individual upper and lower tablet weight limits of ±10% (135.0 mg to 165 mg).

Friability is determined by Current USP <1216> at the beginning and end of each compression run and is NMT 0.5%. The above tablet compression process yields 150.0 mg tablets±5.0% having an average thickness of 3.67 mm±5% and an average breaking force of 195 Newtons, with an average tablet breaking force limit of from 170 Newtons to 220 Newtons.

Disintegration times are determined using Current USP <701> at the beginning and end of each compression batch. Disintegration time is NMT 5 minutes.

A 10 mg tablet core may be prepared similarly, but with a larger tablet punch.

### Example 5b

### Coating of Elvucitabine Tablets

A seal coat, and enteric coat and an overcoat are applied according to Table 5.

TABLE 5

| | 5 mg | | 10 mg | |
|---|---|---|---|---|
| Component | % w/w | mg/tablet | % w/w | mg/tablet |
| Tablet cores | 86.96 | 150 | 90.5 | 300 |
| OPADRY Clear, seal coat | 2.18 | 3.75 | 1.81 | 6.0 |
| Acryl EZE beige, enteric coat | 10.43 | 18 | 7.24 | 24.0 |
| OPADRY clear, over coat | 0.43 | 0.75 | 0.45 | 1.5 |
| Total | 100 | 172.5 | 100 | 331.5 |

The seal coat comprising OPADRY Clear, the enteric coating comprising Acryl EZE®, and the over coat comprising OPADRY clear applied sequentially as aqueous coating suspensions using a coating pan. The tablet cores are preheated to 46° C. (Exhaust air temperature). The pan speed is adjusted to provide adequate tablet flow and the coating suspensions are sprayed onto the tablets at an atomizing air pressure of 18-30 psi; an inlet air temperature of 60-70° C. for the seal coat and over coat, and of 42-50° C. for the enteric coat; an exhaust air temperature of 40 to 50° C. for the seal coat and over coat and 30 to 35° C. for the enteric coat; a spray rate of 15 to 50 ml/min.; and an inlet air flow of 175 to 300 cfm. One of skill in the would understand that the processing parameters for coating are dependent in part upon the size of the batch to be coated and can be adjusted accordingly.

### Example 6

### Additional Common Blends

TABLE 6

| Blend Component | Weight | % w/w | Weight | % w/w |
|---|---|---|---|---|
| Silicified Microcrystalline | 9.650 kg | 83.91 | 10.646 kg | 92.57 |
| Cellulose, | | | | |
| NF (Prosolv 90) | | | | |
| Lactose Monohydrate | 0.995 kg | 8.66 | — | — |
| Sodium Starch Glycolate, | 0.230 kg | 2.00 | 0.230 kg | 2.00 |
| NF, EP | | | | |
| Dipotassium Phosphate | 0.230 kg | 2.00 | 0.230 kg | 2.00 |
| Powder, USP, PE | | | | |
| Magnesium Stearate, | 0.012 kg | 0.10 | 0.012 kg | 0.10 |
| NF, EP (VG) | | | | |
| Elvucitabine, micronized | 0.383 kg | 3.33 | 0.383 kg | 3.33 |
| Totals | 11.5 kg | 100.0 | 11.5 kg | 100.0 |

TABLE 7

| Blend Component | Weight | % w/w | Weight | % w/w |
|---|---|---|---|---|
| Silicified | 10.637 kg | 92.5 | 10.068 kg | 87.55 |
| Microcrystalline | | | | |
| Cellulose, | | | | |
| NF (Prosolv 90) | | | | |
| Sodium Starch Glycolate, | 0.201 kg | 1.75 | 0.345 kg | 3.00 |
| NF, EP | | | | |
| Dipotassium Phosphate | 0.345 kg | 3.00 | 0.460 kg | 4.00 |
| Powder, USP, | | | | |
| PE | | | | |
| Magnesium Stearate, | 0.029 kg | 0.25 | 0.052 kg | 0.45 |
| NF, EP (VG) | | | | |
| Elvucitabine, micronized | 0.288 kg | 2.50 | 0.575 kg | 5.0 |
| Totals | 11.5 kg | 100.0 | 11.5 kg | 100.0 |

From the foregoing it will be appreciated that, although specific embodiments of the invention have been described herein for purposes of illustration, various modifications may be made without deviating from the spirit and scope of the invention.

### Example 7

### Relative Strengths of Spray Dried Lactose, Anhydrous Lactose and MCC Blends with Lactose

The instrumentation used for determining relative strengths of Elvucitabine lactose formulations was identical to that reported in Example 2. A Riva Piccola instrumented tablet press was used in the studies to determine the ejection, take-off and compaction forces. The SMI Director program was used to acquire and generate the graphs and reports described in this example.

The results are presented graphically for ease in interpretation and understanding. All graphs for compaction profiles are normalized using the compaction pressure vs. the resulting tensile strength of the tablet.

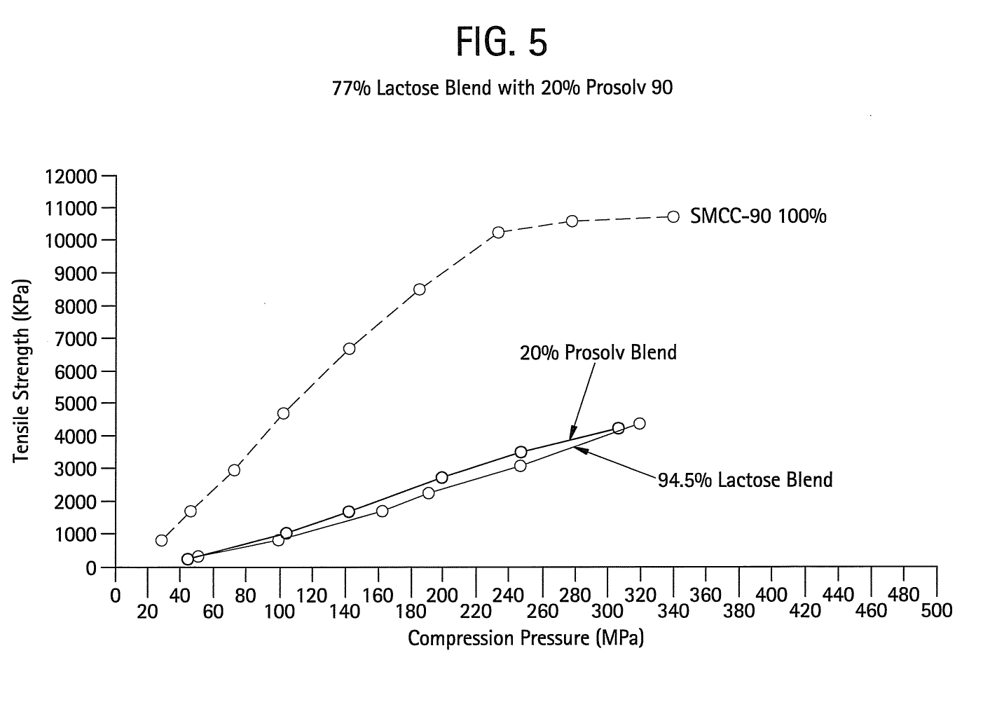

The graph in FIG. 5 demonstrates the strength of the original lactose-based formulation compared to the SMCC-90 blend (The original formulation in which a portion of the lactose is replaced with SMCC-90, 20%). There is essentially no improvement in the tablet strength with the addition of the SMCC-90 into the blend. The large amount of a brittle fracture dominated the lesser amount of plastic deformation to the extent no benefit was obtained with the addition of the plastically deforming material.

The formulations shown in TABLE 8 were used for the original lactose-based formulation and the SMCC-90 blend formulation shown in FIG. 5.

TABLE 8

| Lactose | | Prosolv Blend | |
|---|---|---|---|
| Ingredient | Percent | Ingredient | Percent |
| Lactose Fast Flow 316 | 47.25 | Lactose Fast Flow 316 | 38.675 |
| Lactose Anhydrous | 47.25 | Lactose Anhydrous | 38.675 |
| Calcium Silicate | 2.0 | SMCC-90 | 20.0 |
| Magnesium Stearate | 0.5 | Calcium Silicate | 2.0 |
| Crospovidone | 3.0 | Magnesium Stearate | 2.0 |
| | | Crospovidone | 3.0 |
| Total | 100 | | 100 |

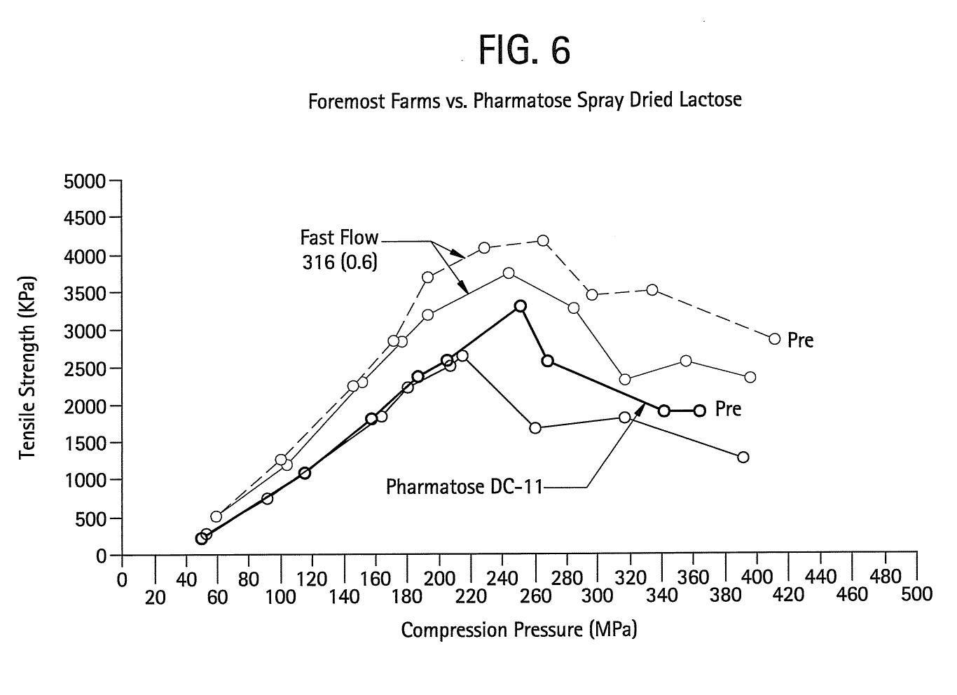

The graph in FIG. 6 represents the results of two suppliers of spray dried lactose, both with and without pre-compression. Note that the pre-compression extends the force the tablet is capable of withstanding before capping. The spray dried lactose supplied by Foremost Farms (Foremost Farms USA, Baraboo, Wis.) showed slightly better compressibility than that from Pharmatose (Pharmatose, BV the Netherlands). For that reason the Foremost Farm Fast Flow 316 product was used in the subsequent studies.

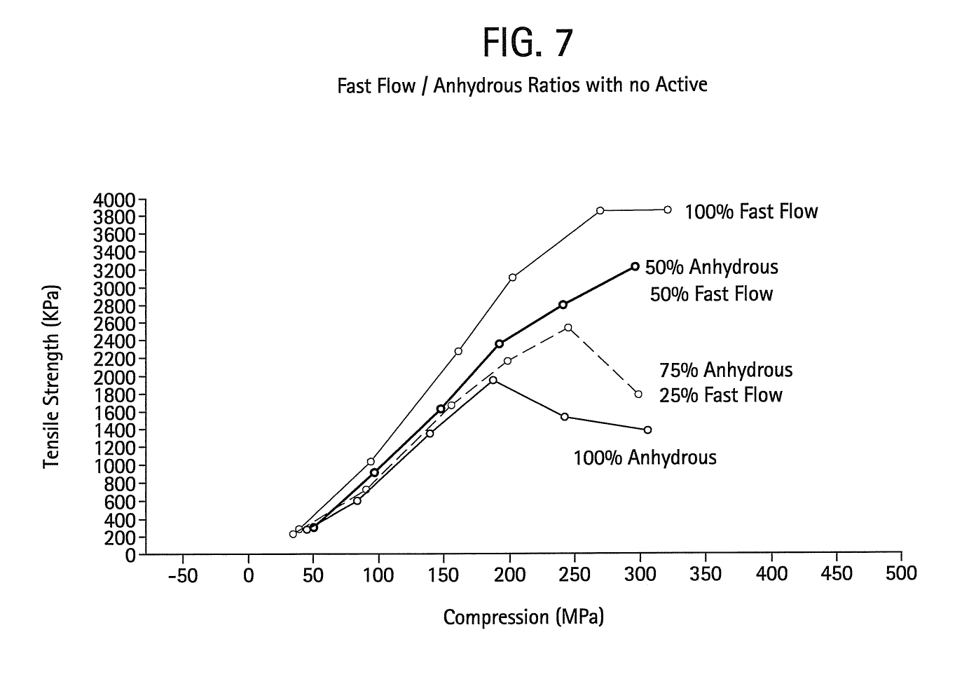

The graph in FIG. 7 shows the results of varying the ratio of the spray dried lactose to the anhydrous lactose. It is clear from the graph in FIG. 7 that as the ratio of the spray dried and anhydrous lactose is increased, the stronger and less likely the compact was to cap. Based on this data, 100% spray dried lactose was used for testing the lactose/SMCC ratios.

During the tabletting phase for the above data it was observed that the spray dried lactose seemed to have more of a sensitivity to the amount of lubricant used than the Anhydrous lactose. Additionally, the strength of the tablet cores using spray-dried lactose was improved with the application of pre-compression. This would lead one to the conclusion that the anhydrous lactose is behaving as a brittle fracture material while the spray dried lactose behaved more plastically. The proper way to verify this observation would be to return to an instrumented single station press to perform additional force displacement tests. This was not done, however, oscilloscope traces of the compression event were taken on the rotary tablet press during the compaction profile so a detailed look at those compressions were investigated.

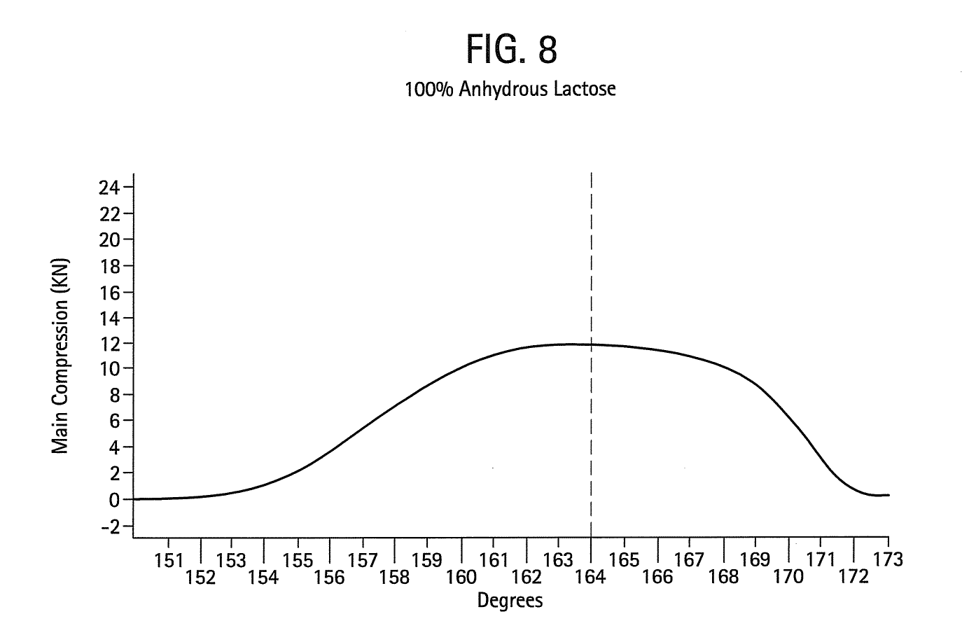

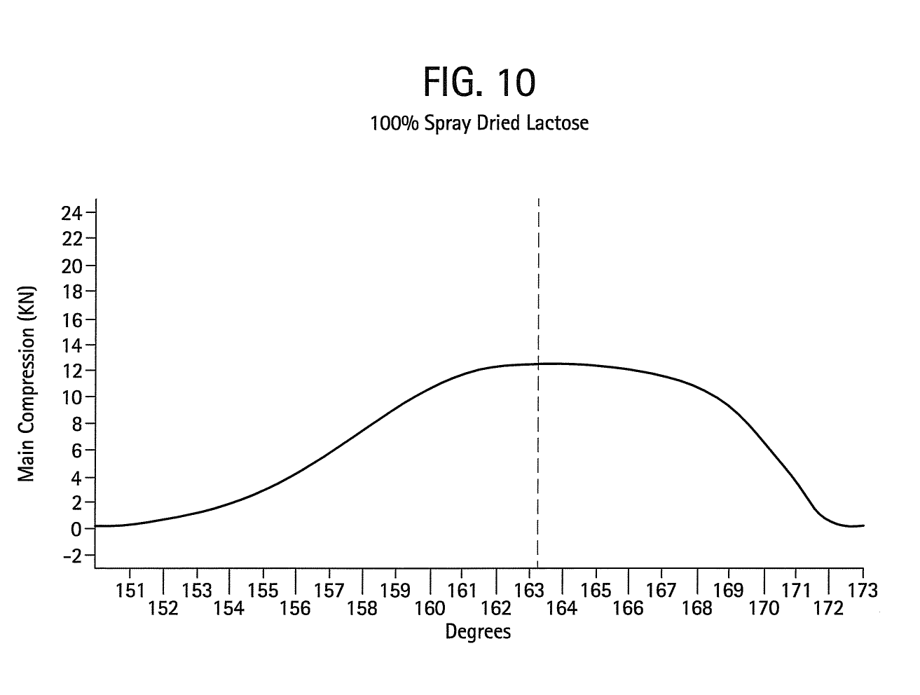

The next three traces in FIGS. 8, 9 and 10 are representative of the compression for the lactose ratios. A compression of a perfectly elastic material would provide a symmetrical curve with equal rise and fall times and equal areas on both sides of the peak force.

Theoretically a completely brittle material compressed on a 100% non-compliant (no stretching) tablet press would resemble the curve above of the perfectly elastic material to the peak force and immediately drop to zero force as the punch head leaves dead center of the compression roll as there would be no elastic expansion of the tablet to maintain the punch head in contact with the roll. The resulting fall time would be very small and there would be no area under the curve after the peak force for the 100% brittle material. Plastic deformation is typically a time dependent visco-elastic recovery, while elastic recovery is immediate. By comparing the ratios of the fall time/rise time and the area from peak force/area to peak the degree of brittleness of the formulation may be ascertained.

These values were calculated using the SMI Director Data Acquisition and Analysis Program. A ratio of zero would be completely brittle; a ratio of 1, perfectly elastic. The following Table provides the summary of values for the 100% anhydrous; 50%/50% and 100% spray dried lactose. The area ratios support the observation that the anhydrous lactose is more brittle than the spray dried, however the fall/rise time ratios show the opposite, although not as strongly.

TABLE 9

| | | 50% Anhydrous | 100% |
|---|---|---|---|
| Ratios | 100% Anhydrous | 50% spray Dried | Spray Dried |
| Area From/To | 0.86 | 0.91 | 1.06 |
| Fall/Rise time | 0.68 | 0.63 | 0.58 |

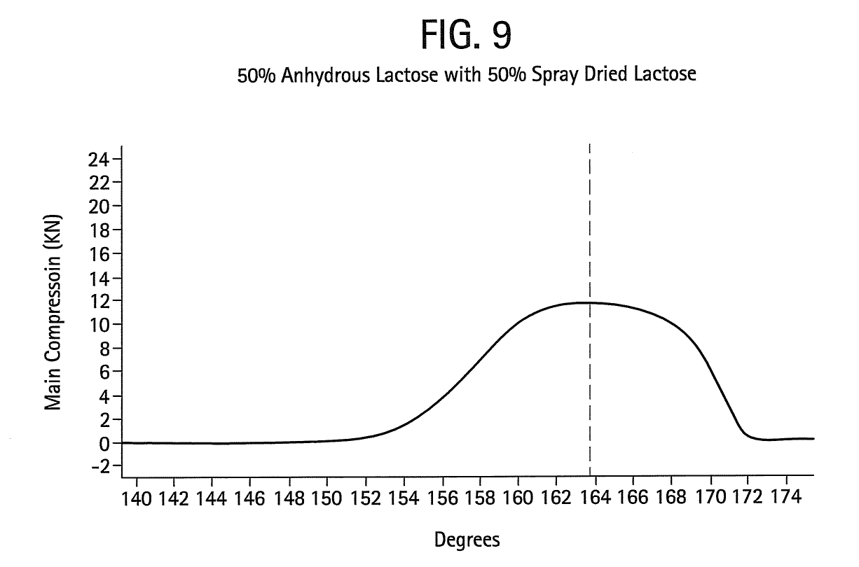

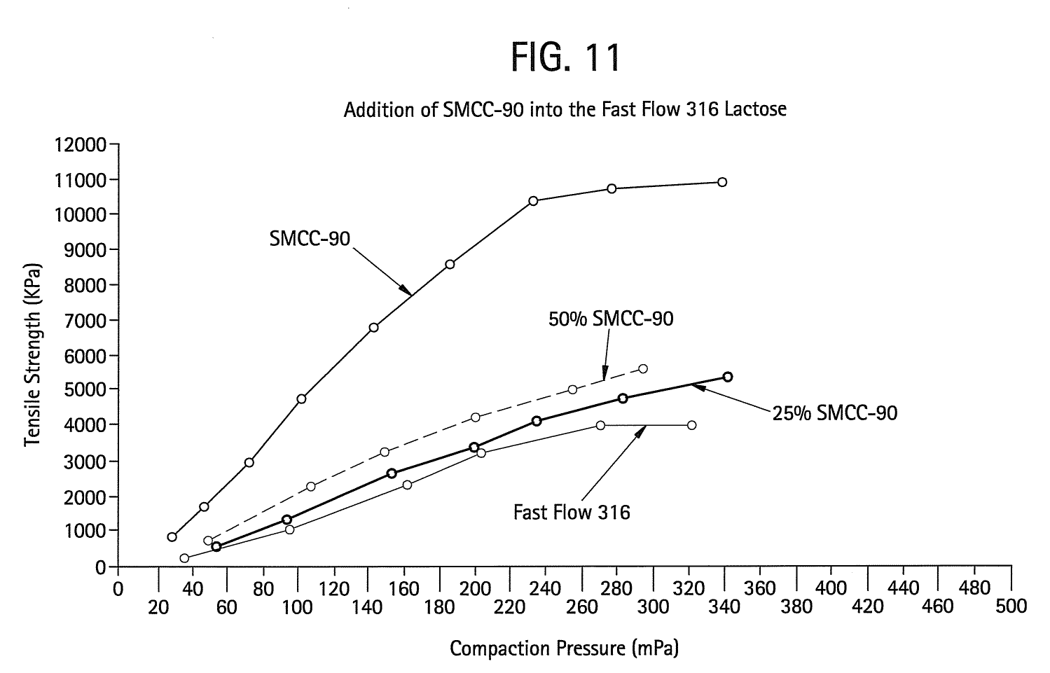

Spray Dried Lactose and SMCC-90 Blend Ratios. Provided that the Foremost Farms Fast Flow 316 is less brittle in its deformation characteristics, it might not dominate the SMCC-90 as did the 50%/50% blend discussed earlier and shown in the graph in FIG. 9. The graph in FIG. 11 shows the impact of 12.5%; 25%, and 50% gradual increase of SMCC-90 into a 100% Foremost Farms Fast Flow 316 blend.

### Example 8

### Additional Elvucitabine Compositions

Formulation 8A. Elvucitabine Core containing 92.89% SMCC-90

TABLE 10
TABLET CORE

| | | Qty. per tablet |
|---|---|---|
| Blend Component | % w/w | (mg) |
| Elvucitabine (micronized) | 3.33 | 10.0 |
| Dipotassium Phosphate Powder, USP, PE | 3.33 | 10.0 |
| Microcrystalline Cellulose and Colloidal | 10.0 | 30.0 |
| Silicon Dioxide, NF (Prosolv 90) Part A | | |
| Microcrystalline Cellulose and Colloidal | 10.0 | 30.0 |
| Silicon Dioxide, NF (Prosolv 90) Part B | | |
| Microcrystalline Cellulose and Colloidal | 62.89 | 188.67 |
| Silicon Dioxide, NF (Prosolv 90) Part C | | |
| Microcrystalline Cellulose and Colloidal | 10.0 | 30.0 |
| Silicon Dioxide, NF (Prosolv 90) Part D | | |
| Starch Sodium Glycolate, NF, EP | 0.25 | 0.75 |
| Magnesium Stearate, NF, EP (VG) (1726) | 0.20 | 0.60 |
| Totals | 100.0 | 300.0 |

TABLE 11
Elvucitabine Coated Table

| | | Qty. per tablet |
|---|---|---|
| Component Description | % w/w | (mg) |
| Elvucitabine Core containing 92.89% | 84.04 | 300.0 |
| SMCC-90 | | |
| OPADRY Orange (SubCoat)¹ | 4.20 | 9.0 |
| ACRYL EZE² | 11.76 | 42.0 |
| OPADRY Orange (OverCoat) | 1.68 | 6.0 |
| Purified Water, USP³ | — | — |
| | 100.0 | 357.0 |

¹OPADRY Orange (Sub Coat before and Over Coat after Enteric Coat) is supplied by Colorcon and is composed of Hypromellose (USP, EP, JP), Iron Oxid Red (NF, JP), Polyethylene Glycol (USP, EP, JP), Titanium Dioxide (USP, FCC, EP, JP), and Iron Oxide Yellow (NF, JP).
²ACRYL EZE is supplied by Colorcon and is composed of Methacrylic Acid Copolymer (USP, EP, JP), Polyethylene Glycol (USP, EP, JP), Silica (NF, EP, JP), Sodium Bicarbonate (USP, EP, JP, FCC, JSFA), Sodium Lauryl Sulfate (NF, EP, JP) & Talc (USP, EP, JP).
³Vehicle for coating solution, evaporated during the coating process

SMCC-90 Parts A and B, Elvucitabine, and Dipotassium Phosphate are blended approximately 10 minutes at 11 rpm. Magnesium stearate, sodium starch glycolate and SMCC-90 Part C are added to the mixture and blended 10 minutes at 11 rpm. Blended material is then passed to a high speed mill with the SMCC-90 Part D. The milled material is screened (30 mesh equivalent) and collected. The milled materials are then blended for an additional time period, approximately 1 hour. The blended material is sampled for particle size and then added to the tablet press. Tablets are pressed to the following specifications shown in TABLE 13:

TABLE 13

| | | Acceptable Upper | Acceptable Lower |
|---|---|---|---|
| Specification | Average | Limit | Limit |
| Average Target | 300.0 mg | 330.0 mg | 270.0 mg |
| Tablet Weight | | (individual | (individual |
| | | upper limit | lower limit) |
| | | 315.0 mg (average | |
| | | upper limit) | |
| Average Target | 4.90 mm | 5.15 mm | 4.65 mm |
| Thickness | | | |
| Average Target | 265 Newtons | 220 Newtons | 176 Newtons |
| Hardness | | | |

| | |
|---|---|
| Friability | NMT 0.5% |
| Disintegration | NMT 5 minutes |

Friability is determined by Current USP <1216> at the beginning and end of each compressing run. Disintegration time is determined as directed by Current USP <701> at the beginning and end of each compressing batch (6 tablets, water at 37±2° C.).

Tablets are coated as follows: OPADRY Orange is mixed for at least 45 minutes with Purified Water, USP (Water: OPADRY Orange approximately 5.7:1.0) until the mixture is smooth and free of lumps and entrapped air. Acryl EZE and purified water (Water: Acryl EZE approximate 4:1) are mixed with a low intensity mixer for at leas 30 minutes until the mixture is smooth, free of lumps and entrapped air. Once mixing is complete the Acryl EZE coating solution is screened through a 60 mesh stainless steel screen.

The coating pan is charged with tablet cores and tablet cores are pre-heated to 46° C. The pan in jogged intermittently while pre-heating tablets. The pan speed is adjusted to provide adequate tablet flow in the coater. Once the solution line is primed with OPADRY Orange solution, the scale is zeroed, and the coating solution sprayed onto the tablets. Tablets are spray coated until and average tablet weight gain of 8.0 to 11.0 mg is achieved. After the initial color coat is applied the solution line is placed into the enteric coating solution and the enteric coating solution is sprayed onto the tablets. Acryl EZE enteric coat is sprayed onto the color coated tablets until the tablets achieve and average weight gain of 42.0 to 48.0 mg. After completion of the enteric coating the solution line is again switched to the color coat. OPADRY Orange is sprayed onto the enterically coated tablets until and average tablet weight gain of 5.0 to 8.0 mg is achieved.

After completion of the final color coat the sprayer is turned off, the pan speed reduced, and the inlet air temperature lowered. The pan is rotated an additional 2 minutes with inlet and exhaust air ON. Cooling continues with frequent jogging of the pan until the Exhaust Air Temp. reaches a minimum of 30° C.

Formulation 8B. Elvucitabine Core containing 92.89% SMCC-90

This formulation is identical to the formulation described in 8A, however the Elvucitabine is uninicronized, resulting in a larger particle size for the API. Elvucitabine is screened, resulting in a particle size of approximately 200 micrometers.

### Formulation 8C. Elvucitabine Core Containing Micronized API and Lactose Monohydrate

TABLE 14
TABLET CORE

| | | Qty. per tablet |
|---|---|---|
| Blend Component | % w/w | (mg) |
| Elvucitabine (micronized) | 3.33 | 10.0 |
| Calcium Silicate, NF | 2.00 | 6.00 |
| Lactose Monohydrate, NF, FastFlo SD316 | 10.0 | 30.0 |
| Part A | | |
| Lactose Monohydrate, NF, FastFlo SD316 | 10.0 | 30.0 |
| Part B | | |
| Lactose Monohydrate, NF, FastFlo SD316 | 61.07 | 183.20 |
| Part C | | |
| Lactose Monohydrate, NF, FastFlo SD316 | 10.0 | 30.0 |
| Part D | | |
| Crospovidone NF | 3.00 | 9.00 |
| Magnesium Stearate, NF, EP (VG) (1726) | 0.60 | 1.80 |
| Totals | 100.0 | 300.0 |

Lactose Monohydrate, NF, FastFlow Parts A and B, Elvucitabine, and Calcium Silicate are blended approximately 10 minutes at 11 rpm. Magnesium stearate, crospovidone NF and Lactose Monohydrate Part C are added to the mixture and blended 10 minutes at 11 rpm. Blended material is then passed to a high speed mill with the Lactose Monohydrate Part D. The milled material is screened (30 mesh equivalent) and collected. The milled materials are then blended for an additional time period, approximately 1 hour. The blended material is sampled for particle size and then added to the tablet press. These tablets are pressed to specifications set forth for the formulation described in Example 8A and according to the procedure set forth for the formulation of Example 8A. Average hardness for a 10 mg of this core formulation is 145-165 Newtons.

This tablet core is coated with the coating composition set forth in Example 8A, Table 11 according to the procedure set forth in Example 8A.

### Formulation 8D. Elvucitabine Core Containing Micronized API and Lactose Monohydrate and Lactose Anhydrous

TABLE 15
TABLET CORE

| | | Qty. per tablet |
|---|---|---|
| Blend Component | % w/w | (mg) |
| Elvucitabine (micronized) | 3.33 | 10.0 |
| Calcium Silicate, NF | 2.00 | 6.00 |
| Lactose Monohydrate, NF, FastFlo SD316 | 10.0 | 30.0 |
| Part A | | |
| Lactose Monohydrate, NF, FastFlo SD316 | 36.07 | 108.20 |
| Part B | | |
| Lactose, NF Anhydrous DT (Direct | 10.0 | 30.0 |
| tableting) Part A | | |
| Lactose, NF Anhydrous DT (Direct | 25.0 | 75.0 |
| tableting) Part B | | |
| Lactose, NF Anhydrous DT (Direct | 10.0 | 30.0 |
| tableting) Part C | | |
| Crospovidone NF | 3.00 | 9.00 |
| Magnesium Stearate, NF, EP (VG) (1726) | 0.60 | 1.80 |
| Totals | 100.0 | 300.0 |

Lactose Monohydrate NF, FastFlo Part A, Elvucitabine, Calcium Silicate, and Lactose NF, Anhydrous DT, Part A are blended approximately 10 minutes at 11 rpm. Magnesium stearate, Crospovidone, Lactose NF, Anhydrous DT, Part B, Lactose Monohydrate NF, FastFlo Part B are added to the mixture and blended 10 minutes at 11 rpm. Blended material is then passed to a high speed mill with the Lactose Monohydrate NF, FastFlo Part C. The milled material is screened (30 mesh equivalent) and collected. Lactose NF, Anhydrous DT, Part C is passed separately through the high-speed mill and screened. The milled materials are then combined and blended at 11 rpm for an additional time period, approximately 1 hour. The blended material is sampled for particle size and then added to the tablet press. Tablets are pressed to the specifications set forth for the formulation described in Example 8A. Tablets are coated as set forth in Example 8A.

### Formulation 8E. Elvucitabine Core Containing 92.89% SMCC-90

This formulation is identical to the formulation described in 8A, however the Elvucitabine is unmicronized, resulting in a larger particle size for the API. The API particle size is approximately 500-600 micrometers.

### Example 9

### Comparative Stability Studies

A systematic evaluation of the physical and biophysical properties of three elvucitabine formulations was performed. The three formulations are the Lactose Fast Flow Formulation (Optimized Lactose) of Example 8C, the PROSOLV formulation of example 8A (Optimized PROSOLV), and the lactose formulation of Example 7, Table 8 (Current Lactose

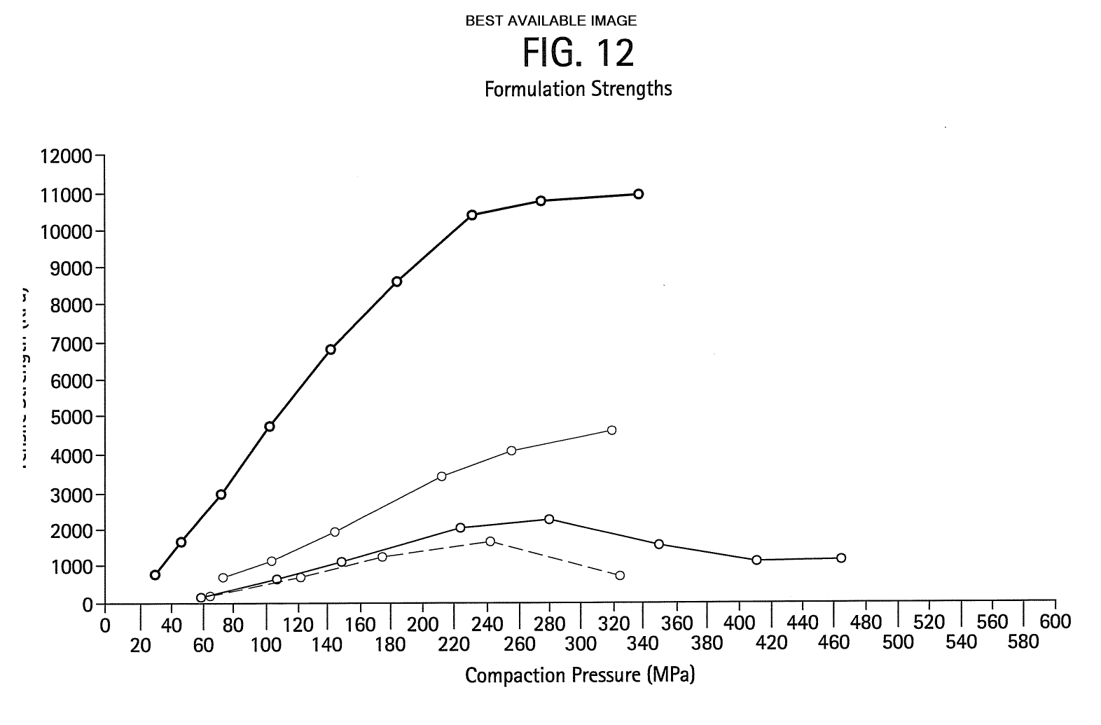

Formulation). Both the optimized Lactose formulation and the Optimized PROSOLV formulations showed improved tensile strength compared to the Current Lactose Formulation as demonstrated by the data in FIG. 12.

### 9A. Blend Uniformity

The use of micronized elvucitabine with particle size less than 200 microns in the Optimized Lactose and Optimized PROSOLV formulations resulted in significant improvement in blend uniformity and tablet dosage uniformity. Content uniformity data obtained via Current USP Protocol <905> for all three formulations is provided in Table 16.

| | | Optimized | Optimized | |
|---|---|---|---|---|
| | | Lactose | PROSOLV | Current |
| | | (Lot | (Lot | Lactose |
| Test | Specification | B06224) | B06222) | (Lot 1321-445) |
| Content | 85.0%-115.0% | 99.7% | 98.5% | 100.2% |
| Uniformity | RSD ≦ 6.0% | RSD 1.3% | RSD 2.2% | RSD 6.6%* |
| USP | | | | |
| <905> | | | | |

*Pass at 2^{nd} level based on 30 tablets; the USP criteria is RSD ≦ 7.8%

### 9B. Formulation Stability

The stability protocol is presented in Table 17. The accelerated and long-term stability examines the effects of temperature and relative humidity on elvucitabine enteric coated (EC) tablets. ICH conditions are used.

The stability storage conditions are 25° C./60% RH, 30° C./65% RH, and 40° C./75% RH for the Current Lactose formulation and 5° C., 25° C./60% RH, 30° C./65% RH, and 40° C./75% RH for the Optimized Lactose and Optimized PROSOLV formulations.

| Time (months) | 0 | 1 | 3 | 6 | 9 | 12 | 18 | 24 | 36 |
|---|---|---|---|---|---|---|---|---|---|
| 5° C. | | X¹ | X¹ | X¹ | X¹ | X¹ | X¹ | X¹ | X¹ |
| 25° C./60% RH | X | X | X | X | X | X | X | X | X |
| 30° C./65% RH | | X² | X² | X² | X² | X² | | | |
| 40° C./75% RH | | X | X | X | | | | | |

¹Reference samples
²Samples from 30° C./65% RH are only tested if a significant change is observed for 40° C./75% RH.

At each time point the samples are tested for appearance, moisture, assay, impurities, and dissolution.

The stability data up to 12 months under ambient (25° C./60% RH) and up to 6 months under accelerated (40° C./75% RH) storage conditions showed good stability for the Optimized Lactose formulation 10 mg EC tablets for both 1-tablet and 30-tablet per bottle packaging configurations with no identifiable trend.

The stability data up to 6 months under ambient (25° C./60% RH) showed good stability for the Optimized PROSOLV formulation 10 mg EC tablets for both 1-tablet and 30-tablet per bottle packaging configurations with no identifiable trend.

The 10 mg EC Optimized PROSOLV formulation tablets exhibited good stability under the accelerated (40° C./75% RH) storage conditions at the initial, 1-month, 2-month and 3-month time points. At 6-month, both the 1-tablet and the 30 tablet/bottle configurations passed the dissolution test at stage 2. The 30 tablet/bottle also had a single impurity of 1.6% at RRT 0.26. At 6-months, under intermediate storage conditions (30° C./65% RH) the impurity level in the 30 tablets/bottle configuration was well below 1.5% and it passed dissolution test at stage 2.

The Current Lactose formulation demonstrated good stability under 6-month accelerated and 36-month ambient storage conditions except the appearance test at the 3-month and 6-month time points under accelerated storage conditions. A slight browning on the tablets' surfaces was observed at these data points.

### Example 10

### Comparative Bioavailability Studies

The purposes of the bioavailability study were as follows: (1) to determine the single-dose relative bioavailability of three enteric-coated table formulations of 10 mg elvucitabine in healthy male subjects; (2) To evaluate the safety and tolerability of single doses of three enteri-coated table formulation of 10 mg elvucitabine in healthy male subjects; and (3) To evaluate the pharmacokinetics of 10 mg elvucitabine in healthy male subjects. Elvucitabine blood levels for the Optimized PROSOLV, Optimized Lactose and Current Lactose formulation were measured every 0.5 hours for 8 hours and then less frequently up to 336 hours. The bioavailability of each tested formulation was comparable to the other tested formulations.

TABLE 18
Pharmacokinetic Parameters (all values are means)

| | | | | AUC_{0-t/} | |
|---|---|---|---|---|---|
| Formulation | AUC₀₋ₜ | AUC_{inf} | AUC₀₋₇₂ | AUC_{inf} | Cmax |
| Optimized | 412.05 | 458.67 | 375.22 | 88.89 | 55.929 |
| PROSOLV | | | | | |
| Optimized | 460.25 | 516.74 | 421.28 | 87.97 | 58.194 |
| Lactose | | | | | |
| Current Lactose | 411.20 | 466.24 | 369.90 | 86.20 | 47.386 |

Pharmacokinetic Parameters

| Formulation | tmax (h) | half-life (hrs) | CL (L/h) | Varea (L) |
|---|---|---|---|---|
| Optimized | 3.643 | 53.81 | 32.01 | 1873.2 |
| PROSOLV | | | | |
| Optimized | 3.469 | 63.80 | 25.28 | 1967.8 |
| Lactose | | | | |
| Current Lactose | 4.285 | 62.04 | 26.70 | 2178.7 |

What is claimed is:

1. A pharmaceutical composition comprising Elvucitabine and silicified microcrystalline cellulose.

2. The pharmaceutical composition of claim 1, wherein the silicified microcrystalline cellulose comprises about 98% microcrystalline cellulose and about 2% colloidal silicon dioxide, and has a mean particle size of about 90 μm.

3. The pharmaceutical composition of claim 1, comprising a core, wherein the core comprises the Elvucitabine and the silicified microcrystalline cellulose, wherein the core further comprises about 0.5% w/w to about 30% w/w of lactose monohydrate, and wherein the silicified microcrystalline cellulose and the lactose monohydrate together do not comprise more than about 95% w/w of the total weight of the core.

4. A pharmaceutical composition comprising Elvucitabine and lactose monohydrate, wherein substantially all the Elvucitabine is Elvucitabine particles having particle size of less than about 200 microns.

5. The pharmaceutical composition of claim 4, wherein the lactose monhydrate is spray-dried. % w/w

6. The pharmaceutical composition of claim 4, wherein 90% of the Elvucitabine particles have a particle size between 50 and 150 microns.

7. The pharmaceutical composition of any one of claim 4, wherein 98% of the Elvucitabine particles have a particle size between 25 and 180 microns.

8. The pharmaceutical composition claim 4, wherein the composition is an oral dosage form in the form of a tablet.

9. The tablet of claim 8, wherein the tablet has a tensile strength of about 22000 Newtons to about 77000 Newtons.

10. The tablet of claim 8 comprising about 2.5 mg Elvucitabine to about 20 mg Elvucitabine and having a breaking force of about 55 Newtons to about 450 Newtons.

11. The pharmaceutical composition of claim 4, wherein the composition has a compression range of about 80 mPa to about 340 mPa.

12. The tablet of claim 8, wherein the tablet is coated with an enteric coating and the enteric coating comprises a methacrylic acid copolymer.

13. The pharmaceutical composition of claim 4, comprising a core, wherein the core comprises the Elvucitabine and lactose monohydrate, wherein the core comprises at least 85% w/w lactose monohydrate, and wherein the lactose monohydrate and elvucitabine together do not comprise more than 96% w/w of the total weight of the core.

14. The pharmaceutical composition of any one of claim 13, wherein the core has an inverse aspect ratio of 0.45 to 0.6.

15. A method of preparing an Elvucitabine-containing tablet core comprising
providing a sample of Elvucitabine particles in which substantially all particles below 200 μm,
blending the Elvucitabine sample and lactose monohydrate to form an Elvucitabine blend comprising about 1% w/w to about 10% w/w of the Elvucitabine and about 30% w/w to about 95% w/w lactose monohydrate, and
compressing the blend to form a tablet core.

16. The method of claim 15, wherein the Elvucitabine blend additionally comprises about 0.05 to about 2% w/w of the composition is magnesium stearate.

17. The method of claim 16, additionally comprising blending the Elvucitabine blend containing about 0.05 to about 2% w/w magnesium stearate 20 minutes or longer.

18. An Elvucitabine composition comprising Elvucitabine and about 0.05 to about 0.75% w/w magnesium sterate, wherein the composition is blended 30 minutes or longer.
